(19) United States
(12) Patent Application Publication
(10) Pub. No.: US 2005/0129779 A1
(43) Pub. Date: Jun. 16, 2005

(54) Use of a hsp inducing compound to limit the side effects of retinoids

(76) Inventors: Claude Dal Farra, Opio (FR); Nouha Domloge, Valbonne (FR); Dominique Peyronel, Marseille (FR)

Correspondence Address: YOUNG & THOMPSON, 745 SOUTH 23RD STREET, 2ND FLOOR, ARLINGTON, VA, 22202, US

(21) Appl. No.: 10/503,717

(22) PCT Filed: Feb. 5, 2003
(86) PCT No.: PCT/FR03/00359

(30) Foreign Application Priority Data
Feb. 8, 2002 (FR) 02/01539
Feb. 13, 2002 (FR) 02/01746

(51) Int. Cl. A61K 31/7076; A61K 35/12

(52) U.S. Cl. 424/526; 514/45

(57) ABSTRACT

The present invention relates to the use of an efficient quantity of at least a HSP inducing compound in or for the preparation of a composition, the compound or composition being intended to limit the side effects of retinoids. The present invention also relates to a cosmetic, pharmaceutical, or dermatological composition including, at least a retinoid and at least a HSP inducing compound, and its utilisation.

The present invention relates to the use of an effective amount of at least a HSP inducing compound in or for the preparation of a composition, the compound or composition being intended for use in limiting the side effects of retinoids.

The present invention also refers to the use of a cosmetic, pharmaceutical, or dermatological composition composed of at least a retinoid and a HSP inducing compound. The present invention also relates to modes of use of these compositions.

The family of compounds known as retinoids refers to compounds that show a specific profile of biological activity, analogous to that of all-trans retinoic acid (also referred to as vitamin C or Tretinoin) or 9-cis retinoic acid (also called Isotretinoin). These compounds modulate the expression of certain genes through the retinoic acid receptor (RAR receptor).

Cosmetic and/or dermatological compositions based on these retinoids have been significantly developed over the last few years. Their activities have been described, among others, in terms of cellular differentiation and proliferation. Representative compounds of this group are retinoic acid or retinal, and their various isomers.

The pharmaceutical uses of retinoids are very numerous, notably in anti-cancer products. Their use is also common in the treatment of rheumatic, cardiovascular, respiratory, and dermatological afflictions.

Thus, for example, retinoic acid is used dermatologically and cosmetically for the treatment of acne, as is described in U.S. Pat. No. 5,034,228. Moreover, in U.S. Pat. No. 5,719,195, 11-cis retinoic acid is used for treatment of psoriasis.

Additionally, it has been shown that retinoids, in particular retinoic acid, have a favorable effect on the appearance and the general condition of skin as well as enabling the struggle against the signs of aging. For example, a method for combating photo-aging was described in patent EP 0 230498, as well as in patent EP 027404, where 13-cis retinoic acid was used to treat aged skin.

However, it is clearly established that the use of retinoids causes numerous aggravating side effects. In particular, their use is extremely irritating for skin, and it provokes inflammatory phenomena, among other side effects.

Although several methods and formulations have been proposed to reduce these side effects resulting from retinoid use, no solution has actually been completely satisfactory—thus the need for a new product that exercises an action on the side effects resulting from retinoid treatment remains.

The applicant has unexpectedly and astonishingly found that an effective quantity of a HSP inducing compound exerts a beneficial action on skin treated with retinoids. In particular, the HSP (Heat Shock Proteins) inducing compound gives skin the ability to avoid the aggravating side effects that are provoked by retinoids.

HSP are one of the most ancient and well-conserved proteins. Homologues exist in all species and in all kingdoms. For example, kDNA, a single gene in bacteria, has become the multi-genetic complex of HSP 70 in Saccharomyces sp., drosophila, and humans.

If HSP appeared very early in the course of evolution, it is a result of the extreme environmental conditions with which organisms were confronted. In addition to preserving their role as protectors from the effects of stress, HSP proteins have taken on new functions throughout evolution, such as transporting proteins within cells.

The essential defense mechanism of all organisms is the conservation of their genome as well as their protein and membrane contents. To this end, HSP, called stress proteins or Heat Shock Proteins, act to protect or repair genetic material, peptides, and proteins that deteriorate during stress. The synthesis of HSP is thus a general means of defense developed by cells in order to combat the great diversity of potential aggressions.

HSP are also called chaperone molecules since they bind other molecules that are either denatured or in the process of biosynthesis. The goal of these molecular interactions is to prevent the aggregation of altered proteins or those in the process of biosynthesis. HSP also help proteins to achieve their three dimensional structure, they eliminate abnormal proteins, and they participate in protein transport from the cytoplasm to the plasma membrane or to various organelles, such as the mitochondria, the endoplasmic reticulum, the lysosomes, and the nucleus.

By interacting with certain proteins, HSP minimize the probability these proteins will interact with other proteins in an inappropriate way. Also, HSP recognize and bind to molecules that are not in their native conformation as a result of being denatured following stress, or when proteins are being synthesized, folded, and assembled, or when a protein is not located in the correct sub-cellular compartment. HSP thus play a primordial role in protection against all the phenomena of cellular stress.

In skin, studies have revealed the presence of HSP 27, HSP 47, HSP 60, HSP 70, and HSP 90. HSP 27 and HSP 90 are expressed in the basal and suprabasal layers of the epidermis and HSP 60 is also expressed in the suprabasal layer while HSP 70 is expressed in the epidermis (Boechncke et coll., 1994). Other studies have demonstrated the presence of HSP 47 in the epidermis as well.

To the knowledge of the applicant, limiting the side effects of retinoids with the use of HSP inducing compound has never been previously described in the previous art.

Thus, an objective of the present invention is the use of an effective quantity of at least a HSP inducing compound in or for the preparation of a compound or composition intended to limit the side effects of retinoids.

The side effects of retinoids that are discussed relate to all of the aggravating effects that result from the administration of compounds in the retinoid family. These can be inflammatory or irritating phenomena. More generally, the term “aggravating side effect” refers to all of the manifestations that can more or less inconvenience individuals that use retinoids. Examples of these effects are redness and itching of the skin.

From now on, the term “Heat shock Proteins (HSP) inducing compound” will be considered as a composition that, when applied on skin or cells, induces the synthesis of HSP (Heat Shock Proteins) or enables the maintenance of an elevated level of HSP. Most of the time, according to this invention, HSP inducing compound enables the augmentation of cellular concentrations in HSP. The HSP inducing compound induces proteins that are part of one or several of the HSP families, including the small HSP (10-30 kDa), HSP 40, HSP 60, HSP 70, HSP 90 and even HSP 100-110.

According to the present invention, the retinoids in question are selected from the group that includes all-trans retinoic acid, 13-cis retinoic acid, retinaldehydes, as well as each of their salt and ester forms.

According to the present invention, retinoid salts are a part of the group of sodium, potassium, ammonium and alcanolamine in C2-C30 salts.

Retinoids related to the present invention do not contain compounds such as vitamin A (or retinol), which is widely used in cosmetic compositions, but in different applications.

According to another advantageous aspect related to the present invention, HSP inducing compound is an extract of Zooplankton, specifically, the species Artemia salina. According to another aspect of the invention, HSP protein inducing compound is obtained from a dormant form of Artemia salina. According to yet another aspect of the invention, HSP protein inducing compound is an extract of Artemia salina containing at least 150 mg/L of diguanosine tetraphosphate.

Another objective of the invention is the use of an effective quantity of at least a HSP inducing compound in or for the preparation of a composition, the compound or the composition being intended to maintain the physiological concentration of HSP in cells and/or in skin during treatment with retinoids.

More particularly, according to the present invention, the compound or the composition enables the maintenance of physiological levels of HSP in aged cells and/or in aged skin when they are treated with retinoids.

The phrase “Physiological concentration of Heat Shock Proteins>> refers to a HSP expression level that does not exceed normal levels, meaning concentrations that are found naturally in cells and/or that the concentration does not induce cytotoxic effects.

The implication of HSP for cellular and cutaneous protection is very significant. In effect, HSP play a primordial role in protecting cells from stress, and thus in re-establishing a physiological concentration of HSP, particularly when cells are treated with retinoids. The compounds related to the invention can make the advantages of retinoids manifest while preventing the attenuation of the skin's natural defenses, thereby reducing the harmful effects of retinoids.

Another objective of the invention is the use of an effective quantity of at least a HSP inducing compound in a composition, the compound or composition being intended to regularize the concentration of inflammatory cytokines during retinoid treatment on cells and/or skin.

Inflammatory cytokines are a class of molecules that play an important role in launching inflammatory phenomena. These include Interleukins, TNF, Interferons, and numerous other related substances.

HSP inducing compound diminishes cytokine concentrations, such as that of interleukin-1, in a way that does not trigger the inflammatory response. The concentration of interleukin is effectively brought back to the basal intracellular rate. Thus, the concentration of inflammatory cytokines, which are clearly over-expressed during retinoic acid treatment, is modulated in association with the HSP inducing compound. This compound therefore enables the diminution of the inflammatory response induced by retinoids.

Another objective of the invention is the use of an effective quantity of at least a HSP inducing compound in a composition or compound, the compound or composition being intended to protect cells and/or skin from external stresses occurring during retinoid therapies.

External stresses can include all types of aggressions to which skin and/or cutaneous cells can be subjected. These aggressions can be of chemical, physical, biological, or thermal origin. Examples of such aggressions are pollution, UV light, heavy metals, friction, water with high limestone concentration, variations in temperature, or even products with irritating qualities such as tensioactive products, preservatives, solvents, or perfumes.

During retinoid treatment, skin and/or cells are much more sensitive to aggressions. Also, they can more easily develop inflammatory reactions when subjected to external stresses. The use of HSP inducing compound enables skin and/or cells to be better protected, thereby enabling a diminution of the immune response resulting from stress. Another objective of the invention is characterized in that the composition has an active ingredient composition of at least a retinoid and HSP inducing compound in an acceptable cosmetic or pharmaceutical medium.

According to the present invention, the composition can be a cosmetic and/or dermatological and/or pharmaceutical composition. According to this invention, the composition is preferably cosmetic or dermatological in nature, since it is aimed at improving the cutaneous appearance and general cutaneous performance of the individual who uses it.

According to the present invention, the composition is preferably a cosmetic and/or dermatological composition adapted for topical cutaneous application through an acceptable cosmetic or dermatological medium.

It is evident that the invention relates to mammals in general, and more specifically, to humans.

According to another aspect related to the present invention, the HSP inducing compound is an extract of zooplankton of the species Artemia salina. More specifically, the Artemia extract is obtained starting from the species Artemia salina in its dormant state, meaning the state in which the organism has suspended its metabolic activity, thereby isolating itself from the external environment.

According to another advantageous aspect related to the invention, the Artemia salina extract contains at least 150 mg/L of diguanosine tetraphosphate.

To give an order of magnitude, in the composition of the invention, the HSP inducing compound can be used at concentrations of 0.0001%-20% of the total weight of the composition, and preferably, it would be used at an amount of 0.001 to 5% of the total weight of the composition.

The concentration of retinoids can be between 0.0001% and 5% of the total weight of the composition, and preferentially retinoids would represent 0.001% to 1% of the total weight of the composition.

According to the present invention, the retinoids in question were selected from the group that includes all-trans retinoic acid, 13-cis retinoic acid, 9-cis-retinoic acid, retinaldehydes as well as their salt and ester forms.

According to another aspect of the present invention, the HSP inducing compound mentioned previously is solubilized beforehand in one or several pharmaceutically or cosmetically acceptable solvents such as water, ethanol, propanol, isopropanol, propylene glycol, butylene glycol, dipropylene glycol, ethoxy diglycol, propoxyl, cyclic polyols, vaseline, vegetable oil, or any combinations of these solvents.

According to another advantageous aspect of the present invention, the HSP inducing compound mentioned previously can be solubilized beforehand in pharmaceutical vectors such as liposomes or it can be adsorbed on powdered organic polymers or in a mineral support such as talc and bentonites. More generally, it can be dissolved in or fixed on all acceptable cosmetic or pharmaceutical vectors.

Whatever form the invention takes, the composition, according to the invention can be ingested, injected, or, applied on skin (on all cutaneous zones of the body), hair, phaners or mucus membranes. According to the mode of administration, the composition related to the invention can be presented under all galenic forms normally used.

The compositions related to the invention are preferably presented under a galenic form adapted for cutaneous topical administration. They cover all the cosmetic and dermatological forms. These compositions must contain an acceptable cosmetic or dermatological medium. That is to say, a medium that is compatible with skin and hair.

These compositions can take the form of an aqueous, hydra-alcoholic, or oil solution in oil-water emulsions, water-oil emulsions or in multiple emulsions. They can also be used as creams, in suspension, or as a powder, as long as it is adapted for application on skin, mucus membranes, lips and/or hair.

These compositions can also be more or less fluid and take the form of creams, lotions, milks, serums, ointments, shampoo, gel, paste and mousse. It can also take a solid form like a stick, or it can be used in aerosols. It can also be used as a skin care product and/or as make-up for skin.

Concerning injection, the composition related to the invention can be an aqueous or oil based lotions or a serum. For application on the eyes, the composition can be used as drops whereas for ingestion it can be used as capsules, granules, syrup or pills.

Moreover, these compositions represent all of the additives that are usually considered for use in this application. These compositions also represent all the possible additives necessary for their formulation such as solvents, thickeners, diluents, anti-oxidants, colorants, solar filters, auto-tanning products, pigments, fillers, preservatives, perfumes, odor absorbers, pharmaceutical and cosmetic active ingredients, essential oils, vitamins, essential fatty acids, tensioactivators, filmogen polymers etc...

In all of these cases, specialists in their field will want to carefully consider the selection of adjuvants, as well as their proportions, so as not to compromise the advantageous properties of the composition relating to the invention. These adjuvants can, for example, correspond to 0.01% to 20% of the total weight of the composition.

When the composition related to the invention is in an emulsion, the fatty phase can represent 5% to 80% by weight, but preferably it would represent 5% to 50% of the weight with respect to the total weight of the composition. Emulsifiers or co-emulsifiers used in the composition are selected among those that are classically used in the domain under consideration. For example, they can be used in a proportion of 0.3% to 30% by weight relative to the total weight of the composition.

Let it be known that specialists in their field should select the complementary compounds for the composition, active or non-active, as well as the amounts of the complementary compounds in such a way that the advantageous properties of the composition will not be perceptibly altered by the envisaged addition.

The compositions related to the present invention can be applied most notably as a cosmetic or pharmaceutical composition for use on skin, mucus membranes and/or semi-mucus membranes. However, they can also be used as a cosmetic composition for hair and/or body hair.

The compositions can be applied particularly in so far as skin protection and skin care products are concerned.

Applications in the domain of facial and body make-up can be considered equally for the compositions of the invention. These applications could include lipstick, foundation, colored creams, as well as sunscreen and artificial tanning compositions.

The compositions of the invention can be used in a great number of treatments, notably cosmetic and dermatological. They can take the form of cosmetic compositions used for skin, lips and/or hair treatment, protection, care and make-up removal and/or cleaning, as well as for make-up applications on skin, lips, eye lashes and/or the body.

The composition relating to the invention can also be applied to pharmaceutical and dermatological compositions for all uses, most notably for topical application.

The composition relating to the invention can also be applied to solid preparations such as soap and other cleaning bar soaps. The composition can also be made in aerosol form in which it can be mixed with pressurized propulsion agents. The composition can also be used orally as toothpaste.

The composition of the invention can be applied as a cosmetic, dermatological or pharmaceutical composition to be used orally. These can take the form of drinkable syrups, tablets, sugarcoated pills, capsules, or even as food and nutritional supplements.

According to the invention, we can add to the composition of the invention, other active agents intended for the prevention and/or treatment of the manifestations of cutaneous aging, and/or protection of skin and/or hair from external aggressions.

Another aspect related to the invention concerns the use of the composition, such as it has been defined previously, to combat, in a curative and/or preventative way, against the manifestations of cutaneous aging.

Manifestations of cutaneous aging include all of the modifications regarding external appearance of skin due to aging. Examples of these modifications include wrinkles and fine lines, limp skin, slackened skin, slimmer looking skin, loss of elasticity and/or skin tone, dull skin, and skin which lacks radiance. It also includes internal skin modifications that do not translate directly as changes in external skin appearance. An example of these internal modifications is the degradation that occurs internally in skin resulting from consecutive exposure to UV radiation.

The composition related to the present invention can also be used to protect cells and/or skin from all types of external aggression to which it can be subjected.

The phrase “external aggression” refers to aggressions produced by the environment. These can be of chemical, physical, biological, or thermal origin. Prime examples of such aggressions are pollution, UV radiation, heavy metals, friction, water with high limestone concentration, variations in temperature as well as products with irritating characteristics such as tensioactive agents, preservatives, solvents and perfumes.

The composition related to the present invention can also be used for all types of traditional skin care applications. Skin care is the process of conserving or re-establishing the healthy functioning of skin or the process of providing the means that serve to preserve or improve the skin's appearance and/or texture.

Thus, skin care refers to hydration, appeasement, and protection against all types of aggression, especially from the sun, as well as the fight to prevent the manifestations of aging, particularly cutaneous manifestations of aging.

The advantages brought about by this composition allow one to serenely consider using retinoids knowing that the skins natural defenses will not be weakened, but rather, that they will be strengthened.

Another aspect related to the present invention concerns a process of cosmetic skin treatment in which the previously defined composition is applied on skin and/or hair in order to limit the side effects of retinoids.

Again, the present invention concerns a process of cosmetic skin treatment meant to treat the signs of cutaneous aging by applying the previously defined composition on skin and/or mucus membranes.

The present invention concerns a process of cosmetic treatment meant to protect skin and/or hair from all types of external aggression by applying the previously defined composition on skin, and/or hair, and/or mucus membranes.

The present invention concerns a process of cosmetic treatment meant to protect skin and hair by applying the previously defined composition on skin and/or, hair, and/or mucus membranes.

The process of cosmetic treatment related to the invention can be implemented most notably by applying the cosmetic compositions here above according to methods habitually used for compositions such as the application of creams, gels, serums, lotions, milks, shampoo, and sun creams, on skin, hair and as a toothpaste applied on the gums.

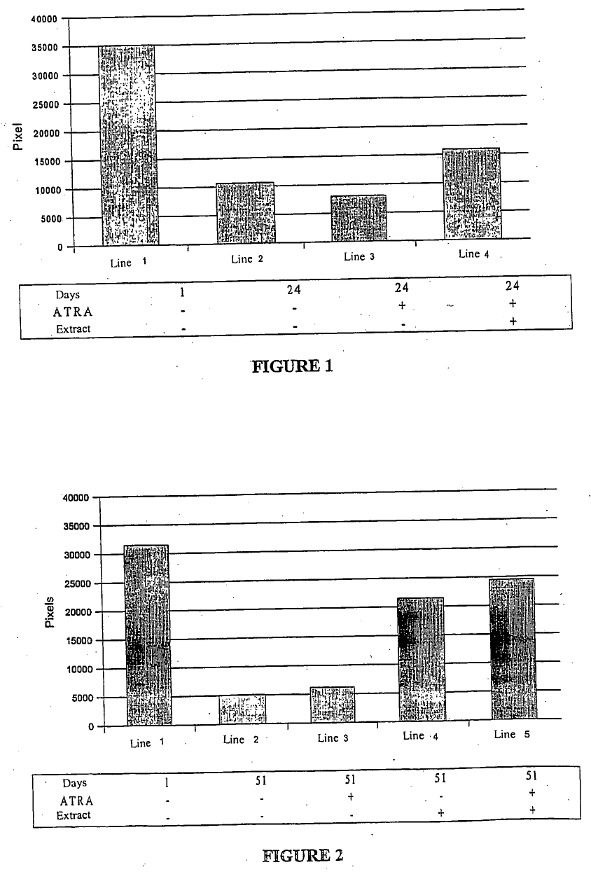

FIG. 1 illustrates the results of an immuno-blot of HSP in human fibroblasts, after one day of culture (line 1) and after 24 days of culture (lines 2, 3, and 4).

FIG. 2 illustrates the results of an immuno-blot of HSP in human fibroblasts after one day of culture (line 1) and after 51 days of culture (lines 2, 3, and 4).

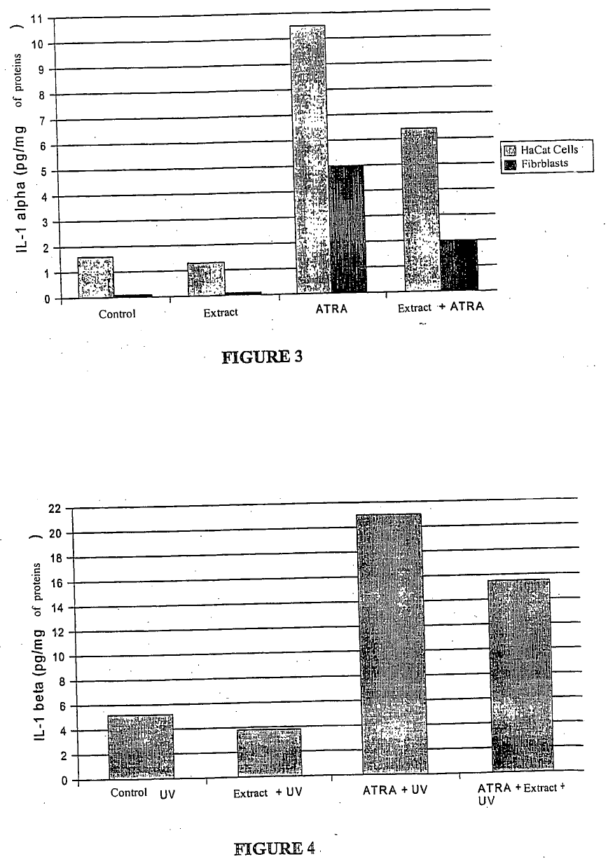

FIG. 3 represents the evaluation of the amount of IL-1 alpha in HaCat cells and in fibroblasts.

FIG. 4 represents an evaluation of the amount of IL-1 Beta in fibroblasts treated with ATRA and or with the extract in example 1, after irradiation with 100 mJ/cm of UVB.

Other advantages and characteristics of the invention will be clarified by reading the following examples, by way of an illustrative and unrestrictive demonstration of data.

### EXAMPLE 1

Protocol for Obtaining a HSP inducing Compound In the first step, 50 grams of zooplankton “eggs” of the species Artemia salina are re-hydrated for between 30 minutes and 6 hours, at between 30° C. to 65° C., in 1 Liter of a medium adapted for the purpose. This medium should be principally composed of water and it should be at a pH of 4-7.

These eggs are then ground in order to obtain a homogenous extract. The intra-cytoplasmic contents are then centrifuged and filtered. Then the extract is sterilized by filtration and heated to 65° C.

HPLC is used to determine if the concentration of diguanosine tetraphosphate is above 150 mg/L.

The extract is then stabilized: it is treated and sterilized so that it conforms to the cosmetic requirements (color, odor, appearance, sterility . . . ). A variable concentration of glycerol is then added to stabilize the proteins. Thus the zooplankton extract contains 0 to 15% glycerol.

The extract is then used in preparations of 0.1% to 15 % of zooplankton extract.

### EXAMPLE 2

The effects of retinoids and the extract from example 1 on the in vivo expression of HSP.

Immuno-blotting studies were conducted on human fibroblasts cultured for 24 and 51 days, respectively, at 37° C. and at 5% CO₂ in a humidified incubator. Several different experimental conditions were used for the fibroblasts.

The conditions that were tested on the fibroblasts were as follows:
Control condition (non-treated cells)
Cells treated with 10⁻⁷M all-trans retinoic acid (ATRA)
Cells treated with 3% of the extract from example 1
Cells treated with 10⁻⁷ M all-trans retinoic acid and 3% of the extract from example 1.

The results illustrated in FIGS. 1 and 2 represent the quantities of HSP obtained by immunoblotting carried out on the human fibroblasts following the treatments in various culture conditions.

The results presented in FIG. 1 demonstrate that in young fibroblasts, meaning cultured for 24 hours, HSP 70 expression is diminished by treatment with retinoic acid. However, the combination of retinoic acid with the extract from example 1 enabled the re-establishment of a physiological HSP concentration.

Moreover, the results in FIG. 2 show that aging fibroblasts, meaning those cultured for 51 hours, express much less HSP 70 than young fibroblasts. In this case, retinoic acid treatment slightly augmented HSP 70 expression, whereas the treatment with the Artemia extract from example 1, both in combination and not in combination with retinoic acid, re-established HSP concentrations close to those of young fibroblasts.

These results also demonstrate that the combination of the extract from example 1 with retinoids enabled the maintenance of physiological HSP concentrations in young and old cells.

### EXAMPLE 3

Effects of Retinoids and the Extract from Example 1 on the Cutaneous Expression of HSP

Ex vivo immunostaining studies have enabled it to be determined that HSP 70 is expressed in skin samples which have been cultured for 5 days. The samples were either treated or untreated with 10⁻⁷retinoic acid plus 1-3% of the extract from example 1. The skin samples were subsequently fixed in 9% formol salt for 10 hours. They were then embedded in paraffin using an automated method. 2-3 urn sections were then cut to be used for immunostaining.

The results obtained demonstrate that skin samples treated with retinoic acid clearly express less HSP 70 than non-treated skin samples. On the other hand, skin treated with a combination of retinoic acid and the extract from example 1 express HSP 70 at a comparable concentration to untreated skin.

Thus, the extract in example 1 allows the re-establishment of a physiological HSP concentration at the cutaneous level. This effect is observed despite the reduction of the HSP concentration caused by retinoic acid treatment.

### EXAMPLE 4

The Effects of Retinoids and the Extract from Example 1 on the Cutaneous Expression of HSP following UV Stress.

The experiments in example three were repeated except that the skin samples were irradiated with a 200 mJ/cm² dose of UVB. The results obtained demonstrated that HSP 70 expression is amplified following UV stress. On the other hand, in the presence of retinoic acid, this expression is clearly diminished, and, to this effect, the skins defenses are reduced. The combination of the extract from example 1 and retinoic acid allowed the skin samples to re-establish their HSP concentration, and thus the skin could protect itself from UV stress.

Although the retinoids diminished the skin sample's defenses against external stresses, the combination with the extract from example 1 gives protection to skin treated with retinoids during stress.

### EXAMPLE 5

The effects of retinoids and the extract from example 1 on the inflammation induced by retinoids.

The Artemia extract from example 1 was used at 3% in in vitro tests in order to show the benefits of applying it in combination with retinoids, with the overall goal of diminishing the inflammation induced by retinoids.

Human HaCat keratinocytes and fibroblasts were cultured in 100mm diameter culture dishes. When the cells reached 50% confluence, they were either treated with 3% of extract from example 1 or not, for 48 hours. In a parallel manner, concentrations of 10⁻⁷M all-trans retinoic acid (ATRA) were, or were not, applied.

The following experimental conditions were used for the different treatment groups:
Control condition (no treatment);
Extract from example 1 for 48n hours;
All-trans retinoic acid (ATRA) for 6 days;
All-trans retinoic acid (ATRA) for 6 days of which 48 hours were treated in combination with the extract from example 1.

After six days in culture, the dose of Interleukin-1 was determined using ELISA, to show evidence of an inflammatory reaction.

The results obtained are illustrated in FIG. 3. They show the dose of Interleukin-1 (IL-1) as a function of the different culture conditions of the experiment. The results demonstrated that while the extract from example 1 does not modulate the inflammatory response, the retinoic acid treatment significantly augmented the IL-1 alpha concentration. In addition, the combination of retinoic acid and the extract from example 1 allowed treated cells to maintain a physiological level of IL-1 alpha.

### Example 6

The effects of Retinoids and the Extract from Example 1 on Inflammation induced by UVB.

Cultured human fibroblasts were subjected to a stress by irradiation using 100 mJ/cm² of UVB.

The irradiated fibroblasts were cultured in different experimental conditions:
Control condition (irradiated cells not treated).
Irradiated cells treated with 3% of the extract in example 1
Irradiated cells treated with 10-7M retinoic acid (ATRA)
Irradiated cells treated with 10-7M-retinoic acid (ATRA) and 3% of the extract from example 1.

After several days, IL-1 beta (alpha was discussed in example 5) dosage was taken using ELISA in order to show evidence of an inflammatory reaction. The results obtained are illustrated in FIG. 4. These results show the dose of IL-1 beta as a function of the different culture conditions that followed the irradiation with 100 mJ/cm² of UVB.

These results demonstrate that the treatment of cells with the extract from example 1 does not modify the inflammatory response of cells that were subjected to UVB radiation. Additionally, these results established that for irradiated cells treated by retinoids and 3% of the extract from example 1, the level of IL-1 beta is lower. Thus, there is a diminution of the inflammatory response.

The HSP inducing compound can thus diminish the inflammatory response induced both by retinoids and UVB radiation. It also protects cells from stresses that originate from external sources during therapies that use retinoids.

### EXAMPLE 7

Preparation of the Composition

This composition is obtained by a simple mix of different compounds. The quantities indicated are given in terms of percentage of weight.

Oil-in-water Emulsion

| | |
|---|---|
| Oil phase: | |
| Montanov 68 (cetearyl Alcohol and Cetearyl Glucoside) | 5.00% |
| Jojoba oi | 5.00% |
| Retinoic Acid | 0.50% |
| Vaseline | 5.00% |
| Isopropyl Palmitate | 7.00% |
| Aqueous phase: | |
| Glycerine | 5.00% |
| Allantoine | 0.10% |
| Extract from example 1 | 5.00% |
| Sepigle 305 (polyacrylamide and C13-14 Isoparaffin and | 0.30% |
| Laureth-7) | |
| Preservative | 0.50% |
| Perfume | 0.50% |
| Water | qs 100% |

1. A method to limit the side effects of retinoids, comprising administering an effective quantity of at least a HSP inducing compound for preparation in a composition.

2. A method to maintain a physiological concentration of HSP, in at least one of cells and skin, during retinoid treatment, comprising administering an effective quantity of at least a HSP inducing compound for preparation in a composition.

3. The method according to claim 2, characterized in that the cells and/or skin are aged.

4. A method to regularize the concentration of inflammatory cytokines in cells and/or skin during treatment with retinoids, comprising administering an effective quantity of at least a HSP inducing compound in or for the preparation of a composition.

5. A method to protect cells and/or skin from external aggressions resulting from retinoid therapy, comprising administering an effective quantity of at least a HSP inducing compound in or for the preparation of a composition.

6. The method of claim 1, characterized in that the HSP inducing compound is an extract of the zooplankton species Artemia salina.

7. The method according to claim 9, wherein the Artemia salina extract is obtained by Artemia salina in a dormant state.

8. The method according to claim 6, wherein the Artemia salina extract contains more than 150 mg/L of diguanosine tetraphosphate.

9. The method according to claim 1 wherein retinoids were selected from among the group of all-trans retinoic acid, 13-cis-retinoic acid, 9-cis-retinoic acid, retinaldehydes, as well as the salts and esters of these molecules.

10. The method according to claim 1, wherein the HSP inducing compound induces HSP that belong to one or several of the families of proteins such as the small HSP (10-30 kDa), HSP 40, HSP60, HSP 70, HSP 90, or HSP 100-110.

11. A cosmetic, pharmaceutical, or dermatological composition including, in an acceptable cosmetic or pharmaceutical medium, at least a retinoid and at least a HSP inducing compound.

12. Composition according to claim 11, characterized in that it takes the form of a cosmetic or dermatological composition adapted for topical cutaneous application.

13. Composition according to claim 11, wherein the HSP inducing compound is an extract of Artemia salina.

14. Composition according to claim 13 characterized in that Artemia salina extract is obtained from the species Artemia salina in its dormant state.

15. Composition according to claim 13, wherein the Artemia salina extract contains at least 150 mg/l of diguanosine tetraphosphate.

16. Composition according to claim 13, characterized in that retinoids are selected from among the group comprising all-trans retinoic acid, 3-cis-retinoc acid, 9-cis-retinoic acid, retinaldehydes, as well as the salt ester forms of these compounds.

17. Composition according to claim 11, wherein the HSP inducing compound is solubilized beforehand in one or more cosmeceutically or pharmaceutically acceptable solvents such as water, vaseline, vegetable oil, propylene glycol, butylene glycol, dipropylene glycol, ethoxylated or propoxylated diglycols, ethanol, propanol or isopropanol.

18. Composition according to claim 11, wherein the HSP inducing compound is solubilized beforehand in a cosmetic or pharmaceutical vector like liposomes or adsorbed on powdered organic polymers and mineral supports like talcs and bentonites.

19. Composition according to claim 11, wherein the concentration of retinoid compounds is between 0.0001 % and 5 % of the total weight of the composition, and preferentially in a quantity representing 0.001 and 1 % of the total weight of the composition.

20. Composition according to claim 11, wherein the HSP inducing compound is present in a quantity representing 0.0001% at 20% of the total weight of the composition.

21. A method of at least one of curing and preventing signs of cutaneous aging, comprising administering an effective amount of the composition claim 11.

22. A method of protecting at least one of cells and skin against all types of aggressions of external origin, comprising administering an effective amount of the composition, defined according to any of claims of claim 11.

23. Process of cosmetic treatment to treat signs of cutaneous aging and/or to protect skin from all types of aggressions of external origin and/or to provide care for skin, comprising applying an effective amount of the cosmetic composition according to claim 11 through 20.
